SEQUENCE LISTING (5 sequences)

SEQ ID NO: 1 - DNA, 2160 bp, Corynebacterium glutamicum
  -35_signal (227)..(232)
  -10_signal (256)..(261)
  CDS (327)..(2063)
ttagaggcgattctgtgaggtcactttttgtggggtcggggtctaaatttggccagttttcgaggcgaccagacaggcgtgcccacgatgtttaaataggcgatcggtgggcatctgtgtttggtttcgacgggctgaaaccaaaccagactgcccagcaacgacggaaatcccaaaagtgggcatccctgtttggtaccgagtacccacccgggcctgaaactccctggcaggcgggcgaagcgtggcaacaactggaatttaagagcacaattgaagtcgcaccaagttaggcaacacaatagccataacgttgaggagttcagatggcacacagctacgcagaacaattaattgacactttggaagctcaaggtgtgaagcgaatttatggtttggtgggtgacagccttaatccgatcgtggatgctgtccgccaatcagatattgagtgggtgcacgttcgaaatgaggaagcggcggcgtttgcagccggtgcggaatcgttgatcactggggagctggcagtatgtgctgcttcttgtggtcctggaaacacacacctgattcagggtctttatgattcgcatcgaaatggtgcgaaggtgttggccatcgctagccatattccgagtgcccagattggttcgacgttcttccaggaaacgcatccggagattttgtttaaggaatgctctggttactgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgcgattggtgcgcaaagtgttgatcgaaaccgccaggtgatcgcgatgtgtggcgatggtggtttgggcatgctgctgggtgagcttctgaccgttaagctgcaccaacttccgctgaaggctgtggtgtttaacaacagttctttgggcatggtgaagttggagatgctcgtggagggacagccagaatttggtactgaccatgaggaagtgaatttcgcagagattgcggcggctgcgggtatcaaatcggtacgcatcaccgatccgaagaaagttcgcgagcagctagctgaggcattggcatatcctggacctgtactgatcgatatcgtcacggatcctaatgcgctgtcgatcccaccaaccatcacgtgggaacaggtcatgggattcagcaaggcggccacccgaaccgtctttggtggaggagtaggagcgatgatcgatctggcccgttcgaacataaggaatattcctactccatgatgattgatacacctgctgttctcattgaccgcgagcgcttaactgccaacatttccaggatggcagctcacgccggtgcccatgagattgccct

SEQ ID NO: 2 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 3 - DNA, 875 bp, Corynebacterium glutamicum
tgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgc

SEQ ID NO: 4 - DNA, 20 bp, Artificial
  Primer
tgcgagatggtgaatggtgg

SEQ ID NO: 5 - DNA, 20 bp, Artificial
  Primer
gcatgaggcaacgcattagc

(19) United States
(12) Patent Application Publication
(10) Pub. No.: US 2005/0196848 A1
(43) Pub. Date: Sep. 8, 2005

(54) Process for the fermentative production of L-amino acids by attenuation of the poxB gene

(76) Inventors: Nicole Dusch, Bielefeld (DE); Brigitte Bathe, Salzkotten (DE); Jorn Kalinowski, Bielefeld (DE); Alfred Puhler, Bielefeld (DE); Bettina Mockel, Bielefeld (DE); Anke Weissenborn, Tubingen (DE); Walter Pfefferle, Halle (DE)

Correspondence Address: PILLSBURY WINTHROP SHAW PITTMAN, LLP, P.O. BOX 10500, MCLEAN, VA, 22102, US

(21) Appl. No.: 11/111,831

(22) Filed: Apr. 22, 2005

Related U.S. Application Data
(62) Division of application No. 09/456,306, filed on Dec. 8, 1999.

(30) Foreign Application Priority Data
Oct. 28, 1999 (DE) 199 51 975.7

(51) Int. Cl. C12Q 1/68; C07H 21/04; C12P 13/08; C12N 1/20; C12N 9/10; C12N 15/74

(52) U.S. Cl. 435/115; 435/6; 435/69.1; 435/193; 435/252.3; 435/471; 536/23.2

(57) ABSTRACT

An isolated polynucleotide containing a polynucleotide sequence selected from the group a) polynucleotide which is at least 70% identical to a polynucleotide which codes for a polypeptide containing the amino acid sequence of SEQ ID no. 2, b) polynucleotide which codes for a polypeptide which contains an amino acid sequence which is at least 70% identical to the amino acid sequence of SEQ ID no. 2, c) polynucleotide which is complementary to the polynucleotides of a) or b) and d) polynucleotide containing at least 15 successive bases of the polynucleotide sequence of a), b) or c), and a process for the fermentative production of L-amino acids by attenuation of the poxB gene.

The present invention provides nucleotide sequences from coryneform bacteria coding for the poxB gene and a process for the fermentative production of amino acids, in particular L-lysine, by attenuation of the poxB gene.

### PRIOR ART

L-amino acids, in particular lysine, are used in human medicine and in the pharmaceuticals industry, in the food industry and very particularly in animal nutrition.

It is known that amino acids are produced by fermentation of strains of coryneform bacteria, in particular Corynebacterium glutamicum. Due to their great significance, efforts are constantly being made to improve the production process. Improvements to the process may relate to measures concerning fermentation technology, for example stirring and oxygen supply, or to the composition of the nutrient media, such as for example sugar concentration during fermentation, or to working up of the product by, for example, ion exchange chromatography, or to the intrinsic performance characteristics of the microorganism itself.

The performance characteristics of these microorganisms are improved using methods of mutagenesis, selection and mutant selection. In this manner, strains are obtained which are resistant to antimetabolites or are auxotrophic for regulatorily significant amino acids and produce amino acids.

For some years, the methods of recombinant DNA technology have also been used for strain improvement of strains of Corynebacterium which produce L-amino acid.

### OBJECT OF THE INVENTION

The inventors set themselves the object of providing novel measures for the improved fermentative production of amino acid, in particular L-lysine.

### DESCRIPTION OF THE INVENTION

L-amino acids, in particular lysine, are used in human medicine and in the pharmaceuticals industry, in the food industry and very particularly in animal nutrition. There is accordingly general interest in providing novel improved process for the production of amino acids, in particular L-lysine.

The present invention provides an isolated polynucleotide containing a polynucleotide sequence selected from the group
a) polynucleotide which is at least 70% identical to a polynucleotide which codes for a polypeptide containing the amino acid sequence of SEQ ID no. 2,
b) polynucleotide which codes for a polypeptide which contains an amino acid sequence which is at least 70% identical to the amino acid sequence of SEQ ID no. 2,
c) polynucleotide which is complementary to the polynucleotides of a) or b) and
d) polynucleotide containing at least 15 successive bases of the polynucleotide sequence of a), b) or c).

The present invention also provides the polynucleotide as claimed in claim 1, wherein it preferably comprises a replicable DNA containing:
(i) the nucleotide sequence shown in SEQ ID no. 1, or
(ii) at least one sequence which matches the sequence (i) within the degeneration range of the genetic code, or
(iii) at least one sequence which hybridises with the complementary sequence to sequence (i) or (ii) and optionally
(iv) functionally neutral sense mutations in (i).

The present invention also provides
a polynucleotide according to claim 2, containing the nucleotide sequence as shown in SEQ ID no. 1,
a polynucleotide as claimed in claim 2 which codes for a polypeptide which contains the amino acid sequence as shown in SEQ ID no. 2,
a vector containing the polynucleotide as claimed in claim 1, point d, in particular pCR2.1poxBint, deposited in E. coli DSM 13114
and coryneform bacteria acting as host cell which contain an insertion or deletion in the pox gene.

The present invention also provides polynucleotides which substantially consist of a polynucleotide sequence, which are obtainable by screening by means of hybridisation of a suitable gene library, which contains the complete gene having the polynucleotide sequence according to SEQ ID no. 1, with a probe which contains the sequence of the stated polynucleotide according to SEQ ID no. 1 or a fragment thereof and isolation of the stated DNA sequence.

Polynucleotide sequences according to the invention are suitable as hybridisation probes for RNA, cDNA and DNA in order to isolate full length cDNA which code for the lrp protein and to isolate such cDNA or genes, the sequence of which exhibits a high level of similarity with that of the pyruvate oxidase gene.

Polynucleotide sequences according to the invention are furthermore suitable as primers for the production of DNA of genes which code for pyruvate oxidase by the polymerase chain reaction (PCR).

Such oligonucleotides acting as probes or primers contain at least 30, preferably at least 20, very particularly preferably at least 15 successive nucleotides. Oligonucleotides having a length of at least 40 or 50 bases are also suitable.

“Isolated” means separated from its natural surroundings.

“Polynucleotide” generally denotes polyribonucleotides and polydeoxyribonucleotides, wherein the RNA or DNA may be unmodified or modified.

“Polypeptides” is taken to mean peptides or proteins which contain two or more amino acids joined via peptide bonds.

The polypeptides according to the invention include a polypeptide according to SEQ ID no. 2, in particular those having the biological activity of pyruvate oxidase and also those which are at least 70%, preferably at least 80% and in particular 90% to 95% identical to the polypeptide according to SEQ ID no. 2 and exhibit the stated activity.

The invention furthermore relates to a process for the fermentative production of amino acids, in particular lysine, using coryneform bacteria, which in particular already produce the amino acids, in particular L-lysine, and in which the nucleotide sequences which code for the poxB gene are attenuated, in particular are expressed at a low level.

In this connection, the term “attenuation” means reducing or suppressing the intracellular activity of one or more enzymes (proteins) in a microorganism, which enzymes are coded by the corresponding DNA, for example by using a weak promoter or a gene or allele which codes for a corresponding enzyme which has a low activity or inactivates the corresponding gene or enzyme (protein) and optionally by combining these measures.

The microorganisms, provided by the present invention, may produce amino acids, in particular lysine, from glucose, sucrose, lactose, fructose, maltose, molasses, starch, cellulose or from glycerol and ethanol. The microorganisms may comprise representatives of the coryneform bacteria in particular of the genus Corynebacterium. Within the genus Corynebacterium, Corynebacterium glutamicum may in particular be mentioned, which is known in specialist circles for its ability to produce L-amino acids.

Suitable strains of the genus Corynebacterium, in particular of the species Corynebacterium glutamicum, are in particular the known wild type strains
Corynebacterium glutamicum ATCC13032
Corynebacterium acetoglutamicum ATCC15806
Corynebacterium acetoacidophilum ATCC13870
Corynebacterium melassecola ATCC17965
Corynebacterium thermoaminogenes FERM BP-1539
Brevibacterium flavum ATCC14067
Brevibacterium lactofermentum ATCC13869 and
Brevibacterium divaricatum ATCC14020
   and amino acid producing mutants or strains produced therefrom, such as for example
   such as for example the L-lysine producing strains
Corynebacterium glutamicum FERM-P 1709
Brevibacterium flavum FERM-P 1708
Brevibacterium lactofermentum FERM-P 1712
Corynebacterium glutamicum FERM-P 6463
Corynebacterium glutamicum FERM-P 6464 and
Corynebacterium glutamicum DSM5714

The inventors succeeded in isolating the novel poxB gene, which codes for the enzyme pyruvate oxidase (EC 1.2.2.2), from C. glutamicum.

The poxB gene or also other genes are isolated from C. glutamicum by initially constructing a gene library of this microorganism in E. coli. The construction of gene libraries is described in generally known textbooks and manuals. Examples which may be mentioned are the textbook by Winnacker, Gene und Klone, Eine Einführung in die Gentechnologie (Verlag Chemie, Weinheim, Germany, 1990) or the manual by Sambrook et al.: Molecular Cloning, A Laboratory Manual (Cold Spring Harbor Laboratory Press, 1989). One very well known gene library is that of E. coli K-12 strain W3110, which was constructed by Kohara et al. (Cell 50, 495-508 (1987)) in λ-vectors. Bathe et al. (Molecular and General Genetics, 252:255-265, 1996) describe a gene library of C. glutamicum ATCC13032, which was constructed using the cosmid vector SuperCos I (Wahl et al., 1987, Proceedings of the National Academy of Sciences USA, 84:2160-2164) in E. coli K-12 strain NM554 (Raleigh et al., 1988, Nucleic Acids Research 16:1563-1575). Börmann et al. (Molecular Microbiology 6(3), 317-326, 1992) also describe a gene library of C. glutamicum ATCC13032, using cosmid pHC79 (Hohn and Collins, Gene 11, 291-298 (1980)). O'Donohue (The Cloning and Molecular Analysis of Four Common Aromatic Amino Acid Biosynthetic Genes from Corynebacterium glutamicum. Ph.D. Thesis, National University of Ireland, Galway, 1997) describes the cloning of C. glutamicum genes using the λ Zap Expression system described by Short et al. (Nucleic Acids Research, 16: 7583).

A gene library of C. glutamicum in E. coli may also be produced using plasmids such as pBR322 (Bolivar, Life Sciences, 25, 807-818 (1979)) or pUC9 (Vieira et al., 1982, Gene, 19:259-268). Suitable hosts are in particular those E. coli strains with restriction and recombination defects, such as for example strain DH5α (Jeffrey H. Miller: “A Short Course in Bacterial Genetics, A Laboratory Manual and Handbook for Escherichia coli and Related Bacteria”, Cold Spring Harbor Laboratory Press, 1992).

The long DNA fragments cloned with the assistance of cosmids or other λ vectors may then in turn be sub-cloned in conventional vectors suitable for DNA sequencing.

DNA sequencing methods are described, inter alia, in Sanger et al. (Proceedings of the National Academy of Sciences of the United States of America USA, 74:5463-5467, 1977).

The resultant. DNA sequences may then be investigated using known algorithms or sequence analysis programs, for example. Staden's program (Nucleic Acids Research 14, 217-232(1986)), Butler's GCG program (Methods of Biochemical Analysis 39, 74-97 (1998)), Pearson & Lipman's FASTA algorithm (Proceedings of the National Academy of Sciences USA 85,2444-2448 (1988)) or Altschul et al.'s BLAST algorithm (Nature Genetics 6, 119-129 (1994)) and compared with the sequence entries available in publicly accessible databases. Publicly accessible nucleotide sequence databases are, for example, the European Molecular Biologies Laboratories database (sic)(EMBL, Heidelberg, Germany) or the National Center for Biotechnology Information database (NCBI, Bethesda, Md., USA).

The novel DNA sequence from C. glutamicum which codes for the poxB gene and, as SEQ ID no. 1, is provided by the present invention, was obtained in this manner. The amino acid sequence of the corresponding protein was furthermore deduced from the above DNA sequence using the methods described above. The resultant amino acid sequence of the poxB gene product is shown in SEQ ID no. 2.

Coding DNA sequences arising from SEQ ID no. 1 due to the degeneracy of the genetic code are also provided by the present invention. DNA sequences which hybridise with SEQ ID no. 1 or parts of SEQ ID no. 1 are similarly provided by the invention. Finally, DNA sequences produced by the polymerase chain reaction (PCR) using primers obtained from SEQ ID no. 1 are also provided by the present invention.

The person skilled in the art may find instructions for identifying DNA sequences by means of hybridisation inter alia in the manual “The DIG System Users Guide for Filter Hybridization” from Boehringer Mannheim GmbH (Mannheim, Germany, 1993) and in Liebl et al. (International Journal of Systematic Bacteriology (1991) 41: 255-260). The person skilled in the art will find instructions for amplifying DNA sequences by means of the polymerase chain reaction (PCR) inter alia in the textbook by Gait, Oligonucleotide synthesis: a practical approach (IRL Press, Oxford, UK, 1984) and in Newton and Graham, PCR (Spektrum Akademischer Verlag, Heidelberg, Germany, 1994).

The inventors discovered that coryneform bacteria produce L-amino acids, in particular L-lysine, in an improved manner once the poxB has been attenuated.

Attenuation may be achieved by reducing or suppressing either expression of the poxB gene or the catalytic properties of the enzyme protein. These measures may optionally be combined.

Reduced gene expression may be achieved by appropriate control of the culture or by genetic modification (mutation) of the signal structures for gene expression. Signal structures for gene expression are, for example, repressor genes, activator genes, operators, promoters, attenuators, ribosome binding sites, the start codon and terminators. The person skilled in the art will find information in this connection for example in patent application WO 96/15246, in Boyd & Murphy (Journal of Bacteriology 170: 5949 (1988)), in Voskuil & Chambliss (Nucleic Acids Research 26: 3548 (1998)), in Jensen & Hammer (Biotechnology and Bioengineering 58: 191 (1998)), in Patek et al. (Microbiology 142: 1297 (1996)) and in known textbooks of genetics and molecular biology, such as for example the textbook by Knippers (“Molekulare Genetik”, 6th edition, Georg Thieme Verlag, Stuttgart, Germany, 1995) or by Winnacker (“Gene und Klone”, VCH Verlagsgesellschaft, Weinheim, Germany, 1990).

Mutations which give rise to a change or reduction in the catalytic properties of enzyme proteins are known from the prior art; examples which may be mentioned are the papers by Qiu and Goodman (Journal of Biological Chemistry 272: 8611-8617 (1997)), Sugimoto et al. (Bioscience Biotechnology and Biochemistry 61: 1760-1762 (1997)) and Mockel (“Die Threonindehydratase aus Corynebacterium glutamicum: Aufhebung der allosterischen Regulation und Struktur des Enzyms”, Berichte des Forschungszentrums Jülichs, Jül-2906, ISSN09442952, Jülich, Germany, 1994). Summary explanations may be found in known textbooks of genetics and molecular biology, such as for example that by Hagemann (“Allgemeine Genetik”, Gustav Fischer Verlag, Stuttgart, 1986).

Mutations which may be considered are transitions, transversions, insertions and deletions. Depending upon the effect of exchanging the amino acids upon enzyme activity, the mutations are known as missense mutations or nonsense mutations. Insertions or deletions of at least one base pair in a gene give rise to frame shift mutations, as a result of which the incorrect amino acids are inserted or translation terminates prematurely. Deletions of two or more codons typically result in a complete breakdown of enzyme activity. Instructions for producing such mutations belong to the prior art and may be found in known textbooks of genetics and molecular biology, such as for example the textbook by Knippers (“Molekulare Genetik”, 6th edition, Georg Thieme Verlag, Stuttgart, Germany, 1995), by Winnacker (“Gene und Klone”, VCH Verlagsgesellschaft, Weinheim, Germany, 1990) or by Hagemann (“Allgemeine Genetik”, Gustav Fischer Verlag, Stuttgart, 1986).

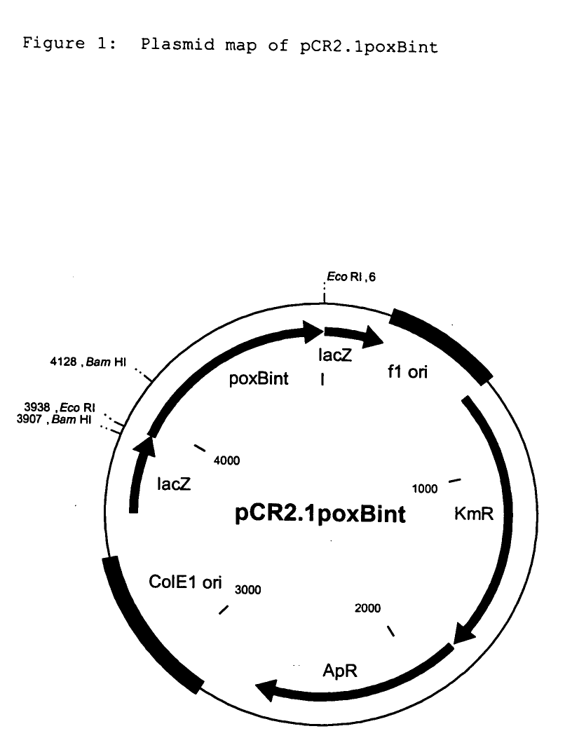

One example of a plasmid with the assistance of which insertion mutagenesis of the poxB gene may be performed is pCR2.1poxBint (FIG. 1).

Plasmid pCR2.1poxBint consists of the plasmid pCR2.1-TOPO described by Mead et al. (Bio/Technology 9:657-663 (1991)), into which an internal fragment of the poxB gene, shown in SEQ ID no. 3, has been incorporated. After transformation and homologous recombination into the chromosomal poxB gene (insertion), this plasmid results in a total loss of enzyme function. By way of example, the strain DSM5715::pCR2.1poxBint, the pyruvate oxidase of which is switched off, was produced in this manner. Further instructions and explanations relating to insertion mutagenesis may be found, for example, in Schwarzer and Pühler (Bio/Technology 9,84-87 (1991)) or Fitzpatrick et al. (Applied Microbiology and Biotechnology 42, 575-580 (1994)).

It may additionally be advantageous for the production of L-amino acids, in particular L-lysine, in addition to attenuating the poxB gene, to amplify, in particular to overexpress, one or more enzymes of the particular biosynthetic pathway, of glycolysis, of anaplerotic metabolism, of the citric acid cycle or of amino acid export.

Thus, for example, for the production of L-lysine
the dapA gene (EP-B 0 197 335) which codes for dihydropicolinate synthase may simultaneously be overexpressed, or
the dapD gene (Wehrmann et al., Journal of Bacteriology 180, 3159-3165 (1998)) which codes for tetradihydropicolinate succinylase may simultaneously be overexpressed, or
the dapE gene (Wehrmann et al., Journal of Bacteriology 177: 5991-5993 (1995)) which codes for succinyldiaminopimelate desuccinylase may simultaneously be overexpressed, or
the gap gene (Eikmanns (1992), Journal of Bacteriology 174:6076-6086) which codes for glyceraldehyde 3-phosphate dehydrogenase may simultaneously be overexpressed, or
the pyc gene (Eikmanns (1992), Journal of Bacteriology 174:6076-6086) which codes for pyruvate carboxylase may simultaneously be overexpressed, or
the mqo gene (Molenaar et al., European Journal of Biochemistry 254, 395-403 (1998)) which codes for malate:quinone oxidoreductase may simultaneously be overexpressed, or
the lysE gene (DE-A-195 48 222) which codes for lysine export may simultaneously be overexpressed.

It may furthermore be advantageous for the production of amino acids, in particular L-lysine, in addition to attenuating the poxB gene, to suppress unwanted secondary reactions (Nakayama: “Breeding of Amino Acid Producing Micro-organisms”, in: Overproduction of Microbial Products, Krumphanzl, Sikyta, Vanek (eds.), Academic Press, London, UK, 1982).

The microorganisms containing the polynucleotide according to claim 1 are also provided by the invention and may be cultured continuously or discontinuously using the batch process or the fed batch process or repeated fed batch process for the purpose of producing L-amino acids, in particular L-lysine. A summary of known culture methods is given in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used must adequately satisfy the requirements of the particular strains. Culture media for various microorganisms are described in “Manual of Methods for General Bacteriology” from the American Society for Bacteriology (Washington D.C., USA, 1981). Carbon sources which may be used include sugars and carbohydrates, such as for example glucose, sucrose, lactose, fructose, maltose, molasses, starch and cellulose, oils and fats, such as for example soya oil, sunflower oil, peanut oil and coconut oil, fatty acids, such as for example palmitic acid, stearic acid and linoleic acid, alcohols, such as for example glycerol and ethanol, and organic acids, such as for example acetic acid. These substances may be used individually or as a mixture. Nitrogen sources which may be used comprise organic compounds containing nitrogen, such as peptones, yeast extract, meat extract, malt extract, corn steep liquor, soya flour and urea or inorganic compounds, such as ammonium sulfate, ammonium chloride, ammonium phosphate, ammonium carbonate and ammonium nitrate. The nitrogen sources may be used individually or as a mixture. Phosphorus sources which may be used are phosphoric acid, potassium dihydrogen phosphate or dipotassium hydrogen phosphate or the corresponding salts containing sodium. The culture medium must furthermore contain metal salts, such as for example magnesium sulfate or iron sulfate, which are necessary for growth. Finally, essential growth-promoting substances such as amino acids and vitamins may also be used in addition to the above-stated substances. Suitable precursors may furthermore be added to the culture medium. The stated materials may be added to the culture in the form of a single batch or may be supplied in a suitable manner during culturing.

Basic compounds, such as sodium hydroxide, potassium hydroxide, ammonia or ammonia water, or acidic compounds, such as phosphoric acid or sulfuric acid, are used appropriately to control the pH of the culture. Antifoaming agents, such as for example fatty acid polyglycol esters, may be used to control foaming. Suitable selectively acting substances, such as for example antibiotics, may be added to the medium in order to maintain plasmid stability. Oxygen or gas mixtures containing oxygen, such as for example air, are introduced into the culture in order to maintain aerobic conditions. The temperature of the culture is normally from 20° C. to 45° C. and preferably from 25° C. to 40° C. The culture is continued until the maximum quantity of the desired amino acid has formed. This objective is normally achieved within 10 hours to 160 hours.

Methods for determining L-amino acids are known from the prior art. Analysis may proceed by anion exchange chromatography with subsequent ninhydrin derivatisation, as described in Spackman et al. (Analytical Chemistry, 30, (1958), 1190) or by reversed phase HPLC, as described in Lindroth et al. (Analytical Chemistry (1979) 51: 1167-1174).

The following microorganism has been deposited with Deutschen Sammlung für Mikrorganismen und Zellkulturen (DSMZ, Braunschweig, Germany) in accordance with the Budapest Treaty:
Escherichia coli strain DH5α/pCR2.1poxBint as DSM 13114.

### EXAMPLES

The present invention is illustrated in greater detail by the following practical examples.

### Example 1

Production of a Genomic Cosmid Gene Library from Corynebacterium glutamicum ATCC13032

Chromosomal DNA from Corynebacterium glutamicum ATCC13032 was isolated as described in Tauch et al., (1995, Plasmid 33:168-179) and partially cleaved with the restriction enzyme Sau3AI (Amersham Pharmacia, Freiburg, Germany, product description Sau3AI, code no. 27-0913-02). The DNA fragments were dephosphorylated with shrimp alkaline phosphatase (Roche Molecular Biochemicals, Mannheim, Germany, product description SAP, code no. 1758250). The DNA of cosmid vector SuperCos1 (Wahl et al. (1987) Proceedings of the National Academy of Sciences USA 84:2160-2164), purchased from Stratagene (La Jolla, USA, product description SuperCos1 Cosmid Vector Kit, code no. 251301) was cleaved with the restriction enzyme XbaI (Amersham Pharmacia, Freiburg, Germany, product description XbaI, Code no. 27-0948-02) and also dephosphorylated with shrimp alkaline phosphatase. The cosmid DNA was then cleaved with the restriction enzyme BamHI (Amersham Pharmacia, Freiburg, Germany, product description BamHI, code no. 27-0868-04). Cosmid DNA treated in this manner was mixed with the treated ATCC 13032 DNA and the batch was treated with T4 DNA ligase (Amersham Pharmacia, Freiburg, Germany, product description T4 DNA Ligase, code no. 27-0870-04). The ligation mixture was then packed in phages using Gigapack II XL Packing Extracts (Stratagene, La Jolla, USA, product description Gigapack II XL Packing Extract, code no. 200217). E. coli strain NM554 (Raleigh et al. 1988, Nucleic Acid Res. 16:1563-1575) was infected by suspending the cells in 10 mM MgSO₄ and mixing them with an aliquot of the phage suspension. The cosmid library was infected and titred as described in Sambrook et al. (1989, Molecular Cloning: A laboratory Manual, Cold Spring Harbor), wherein the cells were plated out on LB agar (Lennox, 1955, Virology, 1:190)+100 μg/ml of ampicillin. After overnight incubation at 37° C., individual recombinant clones were selected.

### Example 2

Isolation and Sequencing of the poxB Gene

Cosmid DNA from an individual colony was isolated in accordance with the manufacturer's instructions using the Qiaprep Spin Miniprep Kit (Product No. 27106, Qiagen, Hilden, Germany) and partially cleaved with the restriction enzyme Sau3AI (Amersham Pharmacia, Freiburg, Germany, product description Sau3AI, code no. 27-0913-02). The DNA fragments were dephosphorylated with shrimp alkaline phosphatase (Roche Molecular Biochemicals, Mannheim, Germany, product description SAP, code no. 1758250). Once separated by gel electrophoresis, the cosmid fragments of a size of approx. 1500 to 2000 bp were isolated using the QiaExII Gel Extraction Kit (product no. 20021, Qiagen, Hilden, Germany). The DNA of the sequencing vector pZero-1 purchased from Invitrogen (Groningen, Netherlands, product description Zero Background Cloning Kit, product no. K2500-01) was cleaved with the restriction enzyme BamHI (Amersham Pharmacia, Freiburg, Germany, product description BamHI, Product No. 27-0868-04). Ligation of the cosmid fragments into the sequencing vector pZero-1 was performed as described by Sambrook et al. (1989, Molecular Cloning: A laboratory Manual, Cold Spring Harbor), wherein the DNA mixture was incubated overnight with T4 ligase (Pharmacia Biotech, Freiburg, Germany). This ligation mixture was then electroporated into the E. coli strain DH5aMCR (Grant, 1990, Proceedings of the National Academy of Sciences U.S.A., 87:4645-4649) (Tauch et al. 1994, FEMS Microbiol Letters, 123:343-7) and plated out onto LB agar (Lennox, 1955, Virology, 1:190)+50 μg/ml of Zeocin. Plasmids of the recombinant clones were prepared using the Biorobot 9600 (product no. 900200, Qiagen, Hilden, Germany, Germany). Sequencing was performed using the dideoxy chain termination method according to Sanger et al. (1977, Proceedings of the National Academies of Sciences U.S.A., 74:5463-5467) as modified by Zimmermann et al. (1990, Nucleic Acids Research, 18:1067). The “RR dRhodamin Terminator Cycle Sequencing Kit” from PE Applied Biosystems (product no. 403044, Weiterstadt, Germany) was used. Separation by gel electrophoresis and analysis of the sequencing reaction was performed in a “Rotiphorese NF” acrylamide/bisacrylamide gel (29:1) (product no. A124.1, Roth, Karlsruhe, Germany) using the “ABI Prism 377” sequencer from PE Applied Biosystems (Weiterstadt, Germany).

The resultant raw sequence data were then processing using the Staden software package (1986, Nucleic Acids Research, 14:217-231), version 97-0. The individual sequences of the pZero 1 derivatives were assembled into a cohesive contig. Computer-aided coding range analysis was performed using XNIP software (Staden, 1986, Nucleic Acids Research, 14:217-231). Further analysis was performed using the “BLAST search programs” (Altschul et al., 1997, Nucleic Acids Research, 25:3389-3402), against the non-redundant database of the “National Center for Biotechnology Information” (NCBI, Bethesda, Md., USA).

The resultant nucleotide sequence is stated in SEQ ID no. 1. Analysis of the nucleotide sequence revealed an open reading frame of 1737 base pairs, which was designated the poxB gene. The poxB gene codes for a polypeptide of 579 amino acids.

### Example 3

Production of an Integration Vector for Integration Mutagenesis of the poxB Gene

Chromosomal DNA was isolated from strain ATCC 13032 using the method of Eikmanns et al. (Microbiology 140: 1817-1828 (1994)). On the basis of the sequence of the poxB gene for C. glutamicum known from Example 2, the following oligonucleotides were selected for the polymerase chain reaction:

poxBint1:
5′ TGC GAG ATG GTG AAT GGT GG 3′
poxBint2:
5′ GCA TGA GGC AAC GCA TTA GC 3′

The stated primers were synthesised by the company MWG Biotech (Ebersberg, Germany) and the PCR reaction performed in accordance with the standard PCR method of Innis et al. (PCR protocols. A guide to methods and applications, 1990, Academic Press) using Pwo polymerase from Boehringer. A DNA fragment of approx. 0.9 kb in size, which bears an internal fragment of the poxB gene and is shown in SEQ ID no. 3, was isolated with the assistance of the polymerase chain reaction.

The amplified DNA fragment was ligated into the vector pCR2.1-TOPO (Mead at al. (1991) Bio/Technology 9:657-663) using the TOPO TA Cloning Kit from Invitrogen Corporation (Carlsbad, Calif., USA; catalogue no. K4500-01). The E. coli strain DH5α was then electroporated with the ligation batch (Hanahan, in DNA cloning. A practical approach. Vol. I. IRL-Press, Oxford, Washington D.C., USA, 1985). Plasmid-bearing cells were selected by plating the transformation batch out onto LB agar (Sambrook et al., Molecular cloning: a laboratory manual. 2^{nd} Ed. Cold Spring Harbor Laboratory Press, Cold Spring Harbor, N.Y., 1989) which had been supplemented with 25 mg/l of kanamycin. Plasmid DN was isolated from a transformant using the QIAprep Spin Miniprep Kit from Qiagen and verified by restriction with the restriction enzyme EcoRI and subsequent agarose gel electrophoresis (0.8%). The plasmid was named pCR2.1poxBint.

### Example 4

Integration Mutagenesis of the poxB Gene into the Lysine Producer DSM 5715

The vector named pCR2.1poxBint in Example 2 was electroporated into Corynebacterium glutamicum DSM 5715 using the electroporation method of Tauch et al. (FEMS Microbiological Letters, 123:343-347 (1994)). Strain DSM 5715 is an AEC-resistant lysine producer. The vector pCR2.1poxBint cannot independently replicate in DSM 5715 and is only retained in the cell if it has been integrated into the chromosome of DSM 5715. Clones with pCR2.1poxBint integrated into the chromosome were selected by plating the electroporation batch out onto LB agar (Sambrook et al., Molecular cloning: a laboratory manual. 2^{nd} Ed. Cold Spring Harbor Laboratory Press, Cold Spring Harbor, N.Y.) which had been supplemented with 15 mg/l of kanamycin. Integration was detected by labelling the poxBint fragment with the Dig hybridisation kit from Boehringer using the method according to “The DIG System Users Guide for Filter Hybridization” from Boehringer Mannheim GmbH (Mannheim, Germany, 1993). Chromosomal DNA of a potential integrant was isolated using the method according to Eikmanns et al. (Microbiology 140: 1817-1828 (1994)) and cut in each case with the restriction enzymes SalI, SacI and HinDIII. The resultant fragments were separated by agarose gel electrophoresis and hybridised at 68° C. using the Dig hybridisation kit from Boehringer. The plasmid named pCR2.1poxBint in Example 3 had been inserted within the chromosomal poxB gene in the chromosome of DSM 5715. The strain was designated DSM5715::pCR2.1poxBint.

### Example 5

Production of Lysine

The C. glutamicum strain DSM5715::pCR2.1poxBint obtained in Example 3 was cultured in a nutrient medium suitable for the production of lysine and the lysine content of the culture supernatant was determined.

To this end, the strain was initially incubated for 24 hours at 33° C. on an agar plate with the appropriate antibiotic (brain/heart agar with kanamycin (25 mg/l)). Starting from this agar plate culture, a preculture was inoculated (10 ml of medium in a 100 ml Erlenmeyer flask). The complete medium CgIII was used as the medium for this preculture. Kanamycin (25 ml/l) was added to this medium. The preculture was incubated for 48 hours at 33° C. on a shaker at 240 rpm. A main culture was inoculated from this preculture, such that the initial optical density (OD, 660 nm) of the main culture was 0.1 OD. Medium MM was used for the main culture.

| Medium MM | | |
|---|---|---|
| CSL (Corn Steep Liquor) | 5 | g/l |
| MOPS | 20 | g/l |
| Glucose (separately autoclaved) | 50 | g/l |
| Salts: | | |
| (NH₄)₂SO₄) | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine*HCl (sterile-filtered) | 0.2 | mg/l |
| Leucine (sterile-filtered) | 0.1 | g/l |
| CaCO₃ | 25 | g/l |

CSL, MOPS and the salt solution are adjusted to pH 7 with ammonia solution and autoclaved. The sterile substrate and vitamin solutions, together with the dry-autoclaved CaCO₃ are then added.

Culturing is performed in a volume of 10 ml in a 100 ml Erlenmeyer flask with flow spoilers. Kanamycin (25 ml/l) was added. Culturing was performed at 33° C. and 80% atmospheric humidity.

After 48 hours, the OD was determined at a measurement wavelength of 660 nm using a Biomek 1000 (Beckmann Instruments GmbH, Munich). The quantity of lysine formed was determined using an amino acid analyser from Eppendorf-BioTronik (Hamburg, Germany) by ion exchange chromatography and post-column derivatisation with ninhydrin detection.

Table 1 shows the result of the test.

TABLE 1

| | | Lysine HCl |
|---|---|---|
| Strain | OD(660) | 5 g/l |
| DSM5715 | 13.1 | 9.5 |
| DSM5715::pCR2.1poxBint | 12.5 | 12.9 |

The following Figures are attached:

FIG. 1: Map of the plasmid pCR2.1poxBint.

The abbreviations and terms used have the following meanings.

| | |
|---|---|
| ColE1 ori: | Replication origin of the plasmid ColE1 |
| lacZ: | 5′ end of the β-galactosidase gene |
| f1 ori: | Replication origin of the f1 phage |
| KmR: | Kanamycin resistance |
| ApR: | Ampicillin resistance |
| BamHI: | Restriction site of the restriction |
| | enzyme BamHI |
| EcoRI: | Restriction site of the restriction |
| | enzyme EcoRI: |
| poxBint2: | Internal fragment of the poxB gene |

1-16. (canceled)

17. A process for the production of an amino acid, comprising the following steps:
a) fermentation of a bacteria producing a desired L-amino acid bacteria, in which at least the poxB gene is attenuated;
b) accumulation of the desired L-amino acid in the medium or in the cells of the bacteria; and
c) isolation of the L-amino acid.

18. The process of claim 17 wherein the amino acid is L-lysine.

19. The process of claim 17, wherein bacteria are used in which further genes of the biosynthetic pathway of the desired L-amino acid are additionally amplified.

20. The process of claim 17, wherein bacteria are used in which the metabolic pathways which reduce the formation of the desired L-amino acid are at least partially suppressed.

21. The process of claim 17, wherein expression of a polynucleotide containing a polynucleotide sequence selected from the group consisting of
a) a polynucleotide which is at least 70% identical to a polynucleotide which encodes a polypeptide containing the amino acid sequence of SEQ ID NO:2,
b) a polynucleotide which encodes a polypeptide which contains an amino acid sequence which is at least 70% identical to the amino acid sequence of SEQ ID NO:2,
c) a polynucleotide which is complementary to the polynucleotides of a) or b), and
d) a polynucleotide containing at least 15 successive bases of the polynucleotide sequence of a), b) or c), is reduced.

22. The process of claim 17, wherein the catalytic properties of a polypeptide containing a polynucleotide sequence selected from the group consisting of
a) a polynucleotide which is at least 70% identical to a polynucleotide which encodes a polypeptide containing the amino acid sequence of SEQ ID NO:2,
b) a polynucleotide which encodes a polypeptide which contains an amino acid sequence which is at least 70% identical to the amino acid sequence of SEQ ID NO:2,
c) a polynucleotide which is complementary to the polynucleotides of a) or b), and
d) a polynucleotide containing at least 15 successive bases of the polynucleotide sequence of a), b) or c), are reduced.

23. The process of claim 17, wherein bacteria are used in which attenuation is achieved by using integration mutagenesis by means of the plasmid pCR2.1poxbint, shown in FIG. 1 and deposited as DSM 13114, or one of the constituents thereof.

24. The process of claim 17, wherein bacteria of the genus Corynebacterium glutamicum are used.

25. A process for the fermentative preparation of L-lysine in Corynebacterium glutamicum comprising:
(a) growing said Corynebacterium glutamicum in which a polynucleotide encoding a PoxB polypeptide of SEQ ID NO: 2 is attenuated by a method of mutagenesis selected from the group consisting of insertion mutagenesis by insertion of at least one base pair, deletion mutagenesis with deletion of at least one base pair, and transition or transversion mutagenesis with incorporation of a non-sense mutation or the activity of said polypeptide is reduced as compared to an unattenuated Corynebacterium glutamicum;
(b) concentrating the L-lysine in the medium or Corynebacterium glutamicum cells; and
(c) isolating said L-amino acid product.

26. The process of claim 25, wherein said polynucleotide is attenuated by integration mutagenesis using plasmid pCR2.1poxBint, as shown in FIG. 1, and deposited as DSM 13114.

27. The process of claim 25, further comprising fermenting C. glutamicum in which one or more genes selected from the group consisting of:
(a) a dapA gene which codes for dihydrodipicolinate synthase;
(b) a pyc gene which encodes pyruvate carboxylase;
(c) a dapE gene which encodes succinyldiaminopimelate desuccinylase;
(d) a dap gene which encodes glyceraldehyde 3-phosphate dehydrogenase;
(e) a mqo gene which encodes a malate:quinone oxidoreductase; and
(f) a lysE gene which encodes a lysine export protein,
are simultaneously over-expressed by increasing the copy number or operatively linking to a heterologous promoter.

28. A process for the fermentative preparation of L-lysine in Corynebacterium comprising:
(a) growing said Corynebacterium in which a Corynebacterium poxB gene is attenuated by a method of mutagenesis selected from the group consisting of insertion mutagenesis by insertion of at least one base pair, deletion mutagenesis with deletion of at least one base pair, and transition or transversion mutagenesis with incorporation of a non-sense mutation or the activity of said polypeptide is reduced as compared to an unattenuated Corynebacterium;
(b) concentrating the L-lysine in the medium or Corynebacterium cells; and
(c) isolating said L-amino acid product.

29. The process of claim 28, wherein said polynucleotide is attenuated by integration mutagenesis using plasmid pCR2.1poxBint, as shown in FIG. 1, and deposited as DSM 13114.

30. The process of claim 29, further comprising fermenting Corynebacterium in which one or more genes selected from the group consisting of:
(a) a dapA gene which codes for dihydrodipicolinate synthase;
(b) a pyc gene which encodes pyruvate carboxylase;
(c) a dapE gene which encodes succinyldiaminopimelate desuccinylase;
(d) a dap gene which encodes glyceraldehyde 3-phosphate dehydrogenase;
(e) a mqo gene which encodes a malate:quinone oxidoreductase; and
(f) a lysE gene which encodes a lysine export protein,
are simultaneously over-expressed by increasing the copy number or operatively linking to a heterologous promoter.
